(19) United States
(12) Patent Application Publication
(10) Pub. No.: US 2018/0272319 A1
(43) Pub. Date: Sep. 27, 2018

(54) PROCESS FOR THE PREPARATION OF A HYDROGENATION CATALYST AND ITS USE FOR THE PREPARATION OF GLYCOLS

(71) Applicant: SHELL OIL COMPANY, HOUSTON, TX (US)

(72) Inventor: Duraisamy MUTHUSAMY, Houston, TX (US)

(21) Appl. No.: 15/763,694

(22) PCT Filed: Sep. 27, 2016
(86) PCT No.: PCT/EP2016/072996

§ 371 Date: Mar. 27, 2018

Related U.S. Application Data
(60) Provisional application No. 62/234,128, filed on Sep. 29, 2015.

(51) Int. Cl. B01J 23/30; C07C 29/132; C07C 29/60

(52) U.S. Cl. CPC B01J 23/30; C07C 31/205; C07C 29/60; C07C 29/132

(57) ABSTRACT

A process for the preparation of an unsupported hydrogenation catalyst wherein a catalyst precursor comprising one or more cations selected from a group consisting of chromium and groups 8, 9, 10 and 11 of the periodic table is contacted in a reactor with hydrazine to convert the catalyst precursor into the unsupported hydrogenation catalyst.

### FIELD OF THE INVENTION

The present invention relates to a process for the preparation of an unsupported hydrogenation catalyst and a process for the preparation of glycols from saccharide-containing feedstocks using the unsupported hydrogenation catalyst.

### BACKGROUND OF THE INVENTION

Glycols such as mono-ethylene glycol (MEG) and mono-propylene glycol (MPG) are valuable materials with a multitude of commercial applications, e.g. as heat transfer media, antifreeze, and precursors to polymers, such as PET. Ethylene and propylene glycols are typically made on an industrial scale by hydrolysis of the corresponding alkylene oxides, which are the oxidation products of ethylene and propylene, produced from fossil fuels.

In recent years, increased efforts have focussed on producing chemicals, including glycols, from non-petrochemical renewable feedstocks, such as sugar-based materials. The conversion of sugars to glycols can be seen as an efficient use of the starting materials with the oxygen atoms remaining intact in the desired product.

Current methods for the conversion of saccharides to glycols revolve around a two-step process of hydrogenolysis and hydrogenation, as described in Angew, Chem. Int. Ed. 2008, 47, 8510-8513.

Such two-step reaction requires at least two catalytic components. Patent application WO2015028398 describes a continuous process for the conversion of a saccharide-containing feedstock into glycols, in which substantially full conversion of the starting material and/or intermediates is achieved and in which the formation of by-products is reduced. In this process the saccharide-containing feedstock is contacted in a reactor vessel with a catalyst composition comprising at least two active catalytic components comprising, as a first active catalyst component with hydrogenation capabilities, one or more materials selected from transition metals from groups 8, 9 or 10 or compounds thereof, and, as a second active catalyst component with retro-aldol catalytic capabilities, one or more materials selected from tungsten, molybdenum and compounds and complexes thereof. Retro-aldol catalytic capabilities referred to herein means the ability of the second active catalyst component to break carbon-carbon bonds of sugars such as glucose to form retro-aldol fragments comprising molecules with carbonyl and hydroxyl groups. Glucose, which is an aldol product, for example, when broken into simple retro-aldol fragments yields glycolaldehyde.

It is well known in the art of chemicals manufacturing that catalysts may be described as homogeneous or heterogeneous, the former being those catalysts which exist and operate in the same phase as the reactants, while the latter are those that do not.

Typically, heterogeneous catalysts may be categorised into two broad groups. One group comprise the supported catalytic compositions where the catalytically active component is attached to a solid support, such as silica, alumina, zirconia, activated carbon or zeolites. Typically these are either mixed with the reactants of the process they catalyse, or they may be fixed or restrained within a reaction vessel and the reactants passed through it, or over it. The other group comprise catalytic compositions where the catalytically active component is unsupported, i.e. it is not attached, to a solid support, an example of this group is the Raney-metal group of catalysts. An example of a Raney-metal catalyst is Raney-nickel, which is a fine-grained solid, composed mostly of nickel derived from a nickel-aluminium alloy. The advantage of heterogeneous catalysts is that they can be retained in the reactor vessel during the process of extracting the unreacted reactants and the products from the reactor vessel, giving the operator the capability of using the same batch of catalysts many times over. However, the disadvantage of heterogeneous catalysts is that over time their activity declines, for reasons such as the loss or leaching of the catalytically active component from its support, or because the access of the reactants to the catalytically active component is hindered due to the irreversible deposition of insoluble debris on the catalyst's support. As their activity declines, catalysts need to be replaced, and for heterogeneous catalysts this inevitably requires the process that they catalyse to be stopped, and the reactor vessel to be opened up, to replace the deactivated catalyst with a fresh batch. Such down-time is costly to the operators of the process, as during such time no products can be produced, and such labour-intensive operations have cost implications.

A further complication of using heterogeneous catalysts is that the process of preparing the catalyst, and in particular the process of immobilising catalytically active components onto a solid support in a way that gives maximum catalytic activity can be difficult and time consuming.

Homogeneous catalysts are typically unsupported and operate in the same phase as the reactants of the reaction they catalyse. Therefore their preparation does not require any step(s) for immobilising the catalytically active components onto a solid support, and their addition to, and mixing with, the reactants of the reaction they catalyse is much easier. However, separation of the catalyst from the reactants becomes more difficult, and in some cases not possible. This means that, in general, homogeneous catalysts either require to be replenished more often than heterogeneous catalysts, and/or additional steps and hardware are required in the process to remove the catalyst from the reactants and reaction products, with an obvious impact on the overall economy of the processes that they catalyse.

Regarding the two-step continuous process of making glycols from saccharide-containing feedstock, as described in WO2015028398, the activities and robustness of the at least two catalytic components, each of which is typically a heterogeneous catalyst, can vary with respect to each other, and therefore if the activity of any one of them declines sooner than the activity of the other, the process of glycol production will not go to completion as efficiently as it was at the commencement of the process, forcing the operators to stop the process to recharge one or both of the catalysts. Alternatively, breakdown components of one of the two catalytic components may adversely affect the other's activity. Again in such a case, the operators of the process are forced to stop the process to recharge one or both of the catalysts. A particular problem is caused by the catalyst component with retro-aldol catalytic capabilities, as over time it degrades and components leach from it. In particular, insoluble tungsten and molybdenum compounds and complexes are formed with the reactant in the reactor vessel over time. This problem is compounded by the deposition of organic degradation products, sintering of metal particles. Such insoluble matter attach to and clog up the surface of the catalyst component with hydrogenation capability, especially if such catalyst component comprises porous solid support and/or is unsupported but nevertheless has a porous surface topology (such as Raney-nickel). Further, the catalyst component with hydrogenation capability may also be poisoning by sulphur or other causes.

It would, therefore be, advantageous to be able to prepare an unsupported hydrogenation catalyst, which is suitable for the hydrogenation of retro-aldol fragments in the process for the preparation of glycols from saccharide-containing feedstock: (i) with minimal labour, including without the time consuming and tricky step of immobilisation of the catalytically active components on a solid support, (ii) which functions with the advantages of both a homogeneous-type and a heterogeneous-type catalysts, but without their respective disadvantages and (iii) which is unaffected by insoluble chemical species originating from the degradation of the catalyst component with retro-aldol catalytic capabilities, so that the two-step process of the conversion of saccharide-containing feedstock to glycols can be carried out in one reaction vessel, thus reducing both capital and operational expenditure associated with the process.

### SUMMARY OF THE INVENTION

The present invention concerns a process for the preparation of an unsupported hydrogenation catalyst, wherein a catalyst precursor comprising one or more cations selected from a group consisting of chromium and groups 8, 9, 10 and 11 of the periodic table is contacted in a reactor with hydrazine to convert the catalyst precursor into the unsupported hydrogenation catalyst.

The present invention also concerns a process for the preparation of glycols from a saccharide-containing feedstock comprising the steps of: (i) preparing an unsupported hydrogenation catalyst by contacting a catalyst precursor comprising one or more elements, selected from chromium and from groups 8, 9, 10 and 11 of the periodic table with hydrazine in a reactor to convert the catalyst precursor into the unsupported hydrogenation catalyst; (ii) preparing in a reactor vessel a reaction mixture comprising the saccharide-containing feedstock, a solvent, a catalyst component with retro-aldol catalytic capabilities and the unsupported hydrogenation catalyst; and (iii) supplying hydrogen gas to the reaction mixture in the reactor vessel.

The inventors of the present processes have surprisingly found that an unsupported hydrogenation catalyst for the production of glycols from a saccharide-containing feedstock can be prepared with minimal labour from a catalyst precursor comprising a cation of an element selected from chromium and groups 8, 9, 10 and 11 of the periodic table. In such preparation, the catalyst precursor is contacted with hydrazine in a reactor to prepare the unsupported hydrogenation catalyst.

The use of hydrazine to prepare the unsupported hydrogenation catalyst provides at least three advantages. The first advantage is that the preparation can be carried out quickly using readily available equipment and reagents. The second advantage is that the unsupported hydrogenation catalyst preparation can be carried out at a lower temperature, and at a lower pressure, than if, for example, hydrogen is used instead of hydrazine. Thirdly, the hydrazine is converted to nitrogen gas during the preparation, and so it can be vented from the reaction mixture. The advantage of this is that the output stream of this process can be directly supplied into the reactor vessel where the production of glycols from a saccharide-containing feedstock is to be, or is being carried out, without the need to undertake any further steps to purify the unsupported hydrogenation catalyst.

### DESCRIPTION OF THE DRAWINGS

FIG. 1 shows a simplified schematic diagram of the embodiment where a single reactor vessel is used for the process for the preparation of glycols from a saccharide-containing feedstock.

FIG. 2 shows a simplified schematic diagram of the embodiment where two reactor vessels are arranged in series are used for the p for the preparation of glycols from a saccharide-containing feedstock.

### DETAILED DESCRIPTION OF THE INVENTION

In the present invention, one or more catalyst precursors is/are contacted in a reactor with hydrazine to convert the catalyst precursor into the unsupported hydrogenation catalyst.

The catalyst precursor is a metal salt or a metal complex. In one embodiment, the catalyst precursor comprises a cation of an element selected from chromium and groups 8, 9, 10 and 11 of the periodic table. Preferably, the cation is selected from the group consisting of chromium, iron, ruthenium, cobalt, rhodium, iridium, nickel, palladium, platinum and copper. More preferably the cation of the salt or metal complex is selected from the group comprising nickel, cobalt and ruthenium. Most preferably, the catalyst precursor comprises a ruthenium cation. In another embodiment, the catalyst precursor comprises a mixture of cations of more than one element selected from chromium and groups 8, 9, 10 and 11 of the periodic table. Preferably, the cations are selected from the group of elements consisting of chromium, iron, ruthenium, cobalt, rhodium, iridium, nickel, palladium, platinum and copper. Suitable examples of such mixture of cations may be a combination of nickel with palladium, or a combination of palladium with platinum, or a combination of nickel with ruthenium, or a combination of chromium with copper.

The catalyst precursor is a metal salt or a metal complex. In one embodiment, the catalyst precursor comprises an anion selected from the group consisting of anions of organic carboxylic acids and any inorganic anion. In the case of both the organic and the inorganic anions, the anion must form a salt or a metal complex with the cations listed above, which is soluble in a mixture comprising the saccharide-containing feedstock, the solvent and the glycols. Preferably, the anion is selected from formate, acetate, oxalate, propionate, lactate, glycolate, stearate, acetylacetonate, nitrate, chloride, bromide, iodide or sulphate. More preferably, the anion is selected from formate, acetate, acetylacetonate and nitrate. Even more preferably, the anion is selected from formate, acetate or acetylacetonate, and most preferably, the anion is formate or acetate. In the embodiment where the catalyst precursor comprises more than one cation, the anion of each of the metal salts or metal complexes may be any one of the anions listed above, with the proviso that each metal salt or each metal complex must be soluble in a mixture comprising the saccharide-containing feedstock, the solvent and the glycols.

In the process of the present invention for the preparation of an unsupported hydrogenation catalyst, an solution of hydrazine is suitably prepared. Preferably the concentration of the hydrazine is at the most 1000 mM, more preferably at the most 500 mM, and most preferably 125 mM. Preferably the concentration of the hydrazine is at least 10 mM, more preferably at least 50 mM, and most preferably at least 75 mM.

In the process of the present invention for the preparation of an unsupported hydrogenation catalyst, a solution of the catalyst precursor is suitably prepared. Preferably, based on the concentration of the cation, the concentration of the catalyst precursor is at the most 1000 mM, more preferably at the most 500 mM, and most preferably 125 mM. Preferably the concentration of the catalyst precursor is at least 10 mM, more preferably at least 50 mM, and most preferably at least 75 mM.

The solution of hydrazine comprises a solvent. Preferably, such solvent is water and/or a solution of glycols in water, and/or the product stream from the reactor vessel used for the process of producing glycols described herein.

The solution of the catalyst precursor comprises a solvent. Preferably, such solvent is water and/or a solution of glycols in water and/or the product stream from the reactor vessel used for the process of producing glycols described herein.

Preferably, the choice of reactors that can be used to carry out such hydrazine treatment of the catalyst precursor are batch reactors, continuous stirred tank reactors (CSTR), pipeline reactors, or a combination comprising a CSTR followed by a pipeline reactor. More preferably, the choice of reactor is a CSTR followed by a pipeline reactor, and most preferably the choice of reactor is a pipeline reactor.

The solution of the catalyst precursor and the solution of hydrazine are pumped into the reactor, and mixed together in the reactor. The ratio of the catalyst precursor to hydrazine pumped into the reactor, on a stoichiometry basis, is preferably at most a ratio of 1.10:1, more preferably at most a ratio of 1.05:1 and most preferably at most a ratio of 1.02:1. The ratio of the solution of the catalyst precursor to the solution of hydrazine pumped into the reactor is preferably at least a ratio of 0.90:1, more preferably at least a ratio of 0.95:1 and most preferably at least a ratio of 0.98:1. The stoichiometric basis of the reduction by hydrazine is 0.5 mole of hydrazine per mole of (2+) charged cation.

In the embodiment where the cation is Ru(3+), the stoichiometric equivalence of hydrazine required to reduce this cation to Ru metal is 0.75 moles of hydrazine per mole of Ru(3+). In the embodiment where the catalyst precursor comprises more than one cation, the ratio of the catalyst precursor to hydrazine pumped into the reactor is calculated on a stoichiometry basis for each cation.

The ratio of the catalyst precursor to hydrazine is important in that, minimal unreacted hydrazine must remain following the hydrazine treatment of the catalyst precursor. As the product of this reaction is supplied directly into the reactor vessel for the preparation of glycols from saccharide-containing feedstock, any unreacted hydrazine that enters the glycol preparation reaction will react with the saccharide-containing feedstock and form hydrazones, which are molecules that do not contribute to the production of glycols. Conversely, insufficient hydrazine will fail to convert the entire catalyst precursor into the unsupported hydrogenation catalyst.

The solution of the catalyst precursor and the solution of hydrazine are preferably maintained in the reactor at a temperature of at least 20° C., more preferably at a temperature of at least 25° C. and most preferably at a temperature of at least 30° C. The solution of the catalyst precursor and the solution of hydrazine are preferably maintained in the reactor at a temperature of at most 230° C., more preferably at a temperature of at most 100° C. and most preferably at a temperature of at most 50° C.

The residence time of the mixture of the solution of the catalyst precursor and the solution of hydrazine in the reactor is preferably at most 60 min, more preferably at most 30 min and most preferably at most 5 min. The residence time of the mixture of the solution of the catalyst precursor and the solution of hydrazine in the reactor is preferably at least 0.1 min, more preferably at least 0.5 min and most preferably at least 1 min.

The output stream obtained from the reactor for contacting the solution of the catalyst precursor with the solution of hydrazine comprises nitrogen gas and the unsupported hydrogenation catalyst. The nitrogen gas is released from this output stream and the remainder of the output stream is pumped into the reactor vessel for the conversion of saccharide-containing feedstock to glycols. Other than the release of the nitrogen gas, no further treatment of the output stream is necessary, however, the output stream becomes acidic during the hydrazine treatment, and if needed, it can be neutralised by any techniques known to the skilled person, such as the addition of sodium hydroxide or sodium carbonate, either during the mixing of the solution of hydrazine with the solution of catalyst precursor, or at a later stage on the output stream itself.

The glycols produced by the process of the present invention are preferably 1,2-butanediol, MEG and MPG, and more preferably MEG and MPG, and most preferably MEG. The mass ratio of MEG to MPG glycols produced by the process of the present invention is preferably 5:1, more preferably 7:1 at 230° C. and 8 MPa.

The saccharide-containing feedstock for the process of the present invention comprises starch. It may also comprise one or further saccharides selected from the group consisting of monosaccharides, disaccharides, oligosaccharides and polysaccharides. An example of a suitable monosaccharide is glucose, and an example of a suitable disaccharide is sucrose. Examples of suitable polysaccharides include cellulose, hemicelluloses, glycogen, chitin and mixtures thereof.

In one embodiment, the saccharide-containing feedstock for said processes is derived from corn. Alternatively, the saccharide-containing feedstock may be derived from grains such as wheat or, barley, from rice and/or from root vegetables such as potatoes, cassava or sugar beet, or any combinations thereof. In another embodiment, a second generation biomass feed such as lignocellulosic biomass, for example corn stover, straw, sugar cane bagasse or energy crops like Miscanthus or sweet sorghum and wood chips, can be used as, or can be part of, the saccharide-containing feedstock.

A pre-treatment step may be applied to the saccharide-containing feedstock to remove particulates and other unwanted insoluble matter, or to render the carbohydrates accessible for hydrolysis and/or other intended conversions. If required, further pre-treatment methods may be applied in order to produce the saccharide-containing feedstock suitable for use in the present invention. One or more such methods may be selected from the group including, but not limited to, sizing, drying, milling, hot water treatment, steam treatment, hydrolysis, pyrolysis, thermal treatment, chemical treatment, biological treatment, saccharification, fermentation and solids removal.

After the pre-treatment, the treated feedstock stream is suitably converted into a solution, a suspension or a slurry in a solvent.

The solvent may be water, or a C₁ to C₆ alcohol or polyalcohol, or mixtures thereof. Suitably C1 to C6 alcohols include methanol, ethanol, 1-propanol and isopropanol. Suitably polyalcohols include glycols, particularly products of the hydrogenation reaction, glycerol, erythritol, threitol, sorbitol, 1,2-hexanediol and mixtures thereof. More suitably, the poly alcohol may be glycerol or 1,2-hexanediol. Preferably, the solvent is water. Further solvent may also be added to a reactor vessel or reactor vessels in a separate feed stream or may be added to the treated feedstock stream before it enters the reactor. Said solvent may be water, or a C₁ to C₆ alcohol or polyalcohol, or mixtures thereof. Suitably C1 to C6 alcohols include methanol, ethanol, 1-propanol and isopropanol. Suitably polyalcohols include glycols, particularly products of the hydrogenation reaction, glycerol, erythritol, threitol, sorbitol, 1,2-hexanediol and mixtures thereof. More suitably, the poly alcohol may be glycerol or 1,2-hexanediol. Preferably, both solvents are the same. More preferably, both solvents comprise water. Most preferably, both solvents are water.

The concentration of the saccharide-containing feedstock as a solution in the solvent supplied to the reactor vessel is at most at 80% wt., more preferably at most at 60% wt. and more preferably at most at 45% wt. The concentration of the saccharide-containing feedstock as a solution in the solvent supplied to the reactor vessel is at least 5% wt., preferably at least 20% wt. and more preferably at least 35% wt.

The unsupported hydrogenation catalyst is prepared using the process discussed above.

The process for the preparation of glycols from a saccharide-containing feedstock requires at least two catalytic components. The first of these is a catalyst component with retro-aldol catalytic capabilities as described in patent application WO2015028398. The role of this catalyst in the glycol production process is to generate retro-aldol fragments comprising molecules with carbonyl and hydroxyl groups from the sugars in the saccharide-containing feedstock, so that the unsupported hydrogenation catalyst can convert the retro-aldol fragments to glycols.

Preferably, the active catalytic components of the catalyst component with retro-aldol catalytic capabilities comprises of one or more compound, complex or elemental material comprising tungsten, molybdenum, vanadium, niobium, chromium, titanium or zirconium. More preferably the active catalytic components of the catalyst component with retro-aldol catalytic capabilities comprises of one or more material selected from the list consisting of tungstic acid, molybdic acid, ammonium tungstate, ammonium metatungstate, sodium metatungstate, ammonium paratungstate, tungstate compounds comprising at least one Group I or II element, metatungstate compounds comprising at least one Group I or II element, paratungstate compounds comprising at least one Group I or II element, heteropoly compounds of tungsten, heteropoly compounds of molybdenum, tungsten oxides, molybdenum oxides, vanadium oxides, metavanadates, chromium oxides, chromium sulphate, titanium ethoxide, zirconium acetate, zirconium carbonate, zirconium hydroxide, niobium oxides, niobium ethoxide, and combinations thereof. The metal component is in a form other than a carbide, nitride, or phosphide. Preferably, the second active catalyst component comprises one or more compound, complex or elemental material selected from those containing tungsten or molybdenum.

In one embodiment, the active catalytic component of the catalyst component with retro-aldol catalytic capabilities is supported on a solid support, and operates as a heterogeneous catalyst. The solid supports may be in the form of a powder or in the form of regular or irregular shapes such as spheres, extrudates, pills, pellets, tablets, monolithic structures. Alternatively, the solid supports may be present as surface coatings, for examples on the surfaces of tubes or heat exchangers. Suitable solid support materials are those known to the skilled person and include, but are not limited to aluminas, silicas, zirconium oxide, magnesium oxide, zinc oxide, titanium oxide, carbon, activated carbon, zeolites, clays, silica alumina and mixtures thereof.

In another embodiment, the catalyst component with retro-aldol catalytic capabilities is unsupported, and operates as a homogeneous catalyst. Preferably, in this embodiment the active catalytic components of the catalyst component with retro-aldol catalytic capabilities is metatungstate, which is delivered into the reactor vessel as an aqueous solution of sodium metatungstate.

The weight ratio of the catalyst component with retro-aldol catalytic capabilities (based on the amount of metal in said composition) to the saccharide-containing feedstock is suitably in the range of from 1:100 to 1:1000.

A reaction mixture comprising the unsupported hydrogenation catalyst, a saccharide-containing feedstock, a solvent, a catalyst component with retro-aldol catalytic capabilities is prepared in the reactor vessel. Said components of the reaction mixture maybe supplied to the reactor vessel in any order.

Preferably, the process of the present reaction takes place in the absence of air or oxygen. In order to achieve this, it is preferable that the atmosphere in the reactor vessel is evacuated and replaced with hydrogen repeatedly, which is carried out after loading of the reaction mixture components, and before the reaction starts. The process of the present invention takes place in the presence of hydrogen. To start the process, the reactor vessel is heated to a reaction temperature and further hydrogen gas is supplied to it under pressure. Hydrogen is supplied into the reactor vessel in a manner common in the art.

Suitable reactor vessels that can be used in the process of the preparation of glycols from a saccharide-containing feedstock include continuous stirred tank reactors (CSTR), plug-flow reactors, slurry reactors, ebullated bed reactors, jet flow reactors, mechanically agitated reactors, bubble columns, such as slurry bubble columns and external recycle loop reactors. The use of these reactor vessels allows dilution of the reaction mixture to an extent that provides high degrees of selectivity to the desired glycol product (mainly ethylene and propylene glycols). In one embodiment, there is a single reactor vessel, which is preferably a CSTR.

There may be more than one reactor vessel used to carry out the process of the present invention. The more than one reactor vessels may be arranged in series, or may be arranged in parallel with respect to each other. In a further embodiment, two reactor vessels arranged in series, preferably the first reactor vessel is a CSTR, the output of which is supplied to a second reactor vessel, which is a plug-flow reactor. The advantage provided by such two reactor vessel embodiment is that the retro-aldol fragments produced in the CSTR have a further opportunity to undergo hydrogenation in the second reactor, thereby increasing the glycol yield of the process.

The weight ratio of the unsupported hydrogenation catalyst (based on the amount of metal in said composition) to the saccharide-containing feedstock is suitably in the range of from 1:100 to 1:1000.

In the embodiment where there is a single reactor vessel used for the process for the preparation of glycols from a saccharide-containing feedstock, the reaction temperature in the reactor vessel is suitably at least 130° C., preferably at least 150° C., more preferably at least 170° C., most preferably at least 190° C. In such embodiment, the temperature in the reactor vessel is suitably at most 300° C., preferably at most 280° C., more preferably at most 270° C., even more preferably at most 250° C. Preferably, the reactor vessel is heated to a temperature within these limits before addition of any starting material and is controlled at such a temperature to facilitate the completion of the reaction.

In the embodiment with a CSTR followed by a plug-flow reactor arranged in series, the reaction temperature in the CSTR is suitably at least 130° C., preferably at least 150° C., more preferably at least 170° C., most preferably at least 190° C. The temperature in the reactor vessel is suitably at most 300° C., preferably at most 280° C., more preferably at most 250° C., even more preferably at most 230° C. In the embodiment with a CSTR followed by a plug-flow reactor arranged in series, the reaction temperature in the plug-flow reactor is suitably at least 50° C., preferably at least 60° C., more preferably at least 80° C., most preferably at least 90° C. The temperature in such reactor vessel is suitably at most 250° C., preferably at most 180° C., more preferably at most 150° C., even more preferably at most 120° C. Preferably, each reactor vessel is heated to a temperature within these limits before addition of any starting material and is controlled at such a temperature to facilitate the completion of the reaction.

In the embodiment where there are two reactor vessels arranged in series, the reaction temperature in each reactor vessel is suitably at least 130° C., preferably at least 150° C., more preferably at least 170° C., most preferably at least 190° C. In such embodiment, the temperature in each reactor vessel is suitably at most 300° C., preferably at most 280° C., more preferably at most 270° C., even more preferably at most 250° C. Preferably, each reactor vessel is heated to a temperature within these limits before addition of any starting material and is controlled at such a temperature to facilitate the completion of the reaction.

In the embodiment where there is a single reactor vessel used for the process for the preparation of glycols from a saccharide-containing feedstock, the pressure in the reactor vessel in which the starting material is contacted with hydrogen in the presence of the catalyst composition described herein is suitably at least 3 MPa, preferably at least 5 MPa, more preferably at least 7 MPa. In such embodiment, the pressure in the reactor vessel is suitably at most 12 MPa, preferably at most 10 MPa, more preferably at most 8 MPa. Preferably, the reactor vessel is pressurised to a pressure within these limits by addition of hydrogen before addition of any starting material and is controlled at such a pressure to facilitate the completion of reaction through on-going addition of hydrogen.

In the embodiment where there are two reactor vessels arranged in series, the pressure in each reactor vessel is suitably at least 3 MPa, preferably at least 5 MPa, more preferably at least 7 MPa. In such embodiment, the pressure in each reactor vessel is suitably at most 12 MPa, preferably at most 10 MPa, more preferably at most 8 MPa. Preferably, each reactor vessel is pressurised to a pressure within these limits by addition of hydrogen before addition of any starting material and is controlled at such a pressure to facilitate the completion of reaction through on-going addition of hydrogen.

In the embodiment where there is a single reactor vessel used for the process for the preparation of glycols from a saccharide-containing feedstock, the residence time in the reactor vessel of the reaction mixture is suitably at least 1 minute, preferably at least 2 minutes, more preferably at least 5 minutes, and suitably the residence time in the reactor vessel is no more than 5 hours, preferably no more than 2 hours, more preferably no more than 1 hour. In the embodiment where there are two reactor vessels arranged in series, the residence time for each of the vessels is suitably at least 1 minute, preferably at least 2 minutes, more preferably at least 5 minutes, and is no more than 5 hours, preferably no more than 2 hours, more preferably no more than 1 hour.

In the embodiment where the catalyst component with retro-aldol catalytic capabilities comprises tungsten supported on a solid support (or a or a combination of solid supports), a problem observed by the inventors of the present application is that the association between tungsten and the solid support is insufficient, leading to leaching of the tungsten from the solid support, and mixing with the other components within the reactor vessel. In the embodiment where the catalyst component with retro-aldol catalytic capabilities comprises unsupported tungsten, by nature of its operation as a homogeneous catalyst, tungsten is in a mixture with the other components within the reactor vessel. In both of these embodiments, the mixture of the tungsten compounds and complexes with the other components within the reactor vessel leads to the formation of insoluble compounds of tungsten, in particular insoluble oxides of tungsten. In particular, the mixture of the tungsten compounds and complexes with saccharide- and glycol-containing aqueous mixtures forms insoluble compounds of tungsten. Such insoluble compounds of tungsten are observed to stick to the pores of solid supports such as silica, alumina, zirconia, activated carbon or zeolites, as well as to the surface of other nano- and micro-entities with rough surface topologies. Where the insoluble compounds of tungsten stick to such pores or surfaces of catalytic entities, they irreversibly reduce the catalytic activity of the catalytic entities by preventing access of the reactants to the surface of the catalytic entity.

The inventors believe that the unsupported hydrogenation catalyst comprises catalytically active micron-sized metal particles. They further believe that the surface topology of the catalytically active micron-sized particles does not contain any significant pores inside the particles, making the unsupported hydrogenation catalyst resistant to the attachment of insoluble chemical species originating from the catalyst component with retro-aldol catalytic capabilities during the process for the preparation of glycols from a saccharide-containing feedstock.

Additionally, the unsupported hydrogenation catalyst produced by the process of the present invention can be handled as if it is a homogeneous catalyst, for example by supplying it into the reactor vessel at the same time as the saccharide-containing feedstock and the solvent.

Whilst having these homogenous-like properties, the unsupported hydrogenation catalyst produced by the process of the present invention can also be handled as if it is a heterogeneous catalyst, for example it can be restrained in the reactor vessel and can be easily separated from the product stream.

Furthermore, no appreciable amounts of hydrazine or any other chemical species that might adversely affect glycol production yields remain in the output stream obtained from the reactor. This means that the output stream obtained from the reactor following the hydrazine treatment of the catalyst precursor can be fed directly into the glycol production reaction without the production of unwanted compounds such as hydrazones in the reactor vessel.

The catalyst precursor can also be supplied into the reactor vessel at any time during the glycol production, enabling the operators to boost any decline in the hydrogenation activity whilst the glycol production is ongoing.

A combined advantage of the abovementioned features is that a simpler and cheaper reactor design and setup can be deployed to carry out the processes of the present invention, for example, it overcomes the need to have any complicated means for catalyst introduction into the reactor vessel. Further, the unsupported hydrogenation catalyst is retained in the reactor vessel by a simple filtration step, therefore otherwise cumbersome solids handling and recovery of deactivated hydrogenation catalyst is solved, and reactor vessels designed for handling homogeneous liquids can be used, and the process of hydrogenation catalyst preparation is significantly simplified.

### DETAILED DESCRIPTION OF THE DRAWINGS

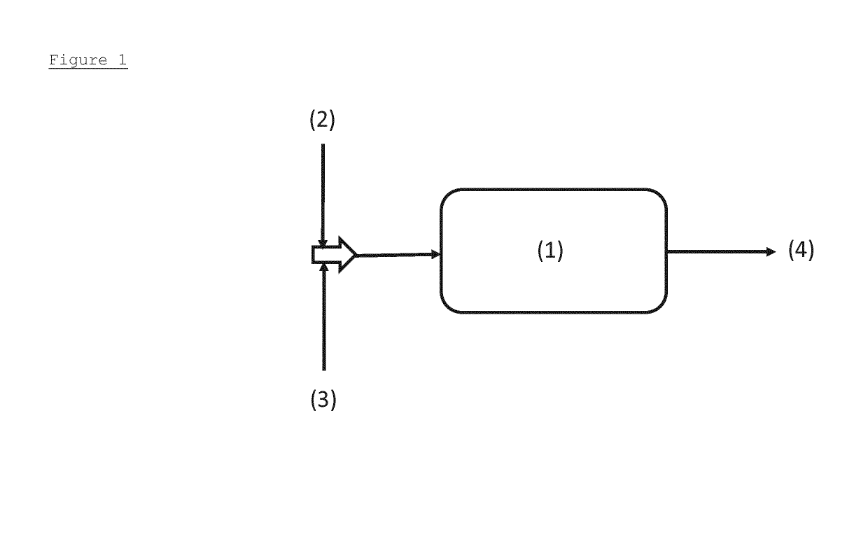

FIG. 1 shows a simplified schematic diagram of the embodiment where a single reactor vessel (1) is used for the process for the preparation of glycols from a saccharide-containing feedstock. A reaction mixture (2) comprising a saccharide-containing feedstock, a solvent and a catalyst component with retro-aldol catalytic capabilities, and hydrogen gas, is supplied to reactor vessel (1), together with the unsupported hydrogenation catalyst (3). The product of the process comprising glycols (4) is obtained as the outflow from reactor vessel (1).

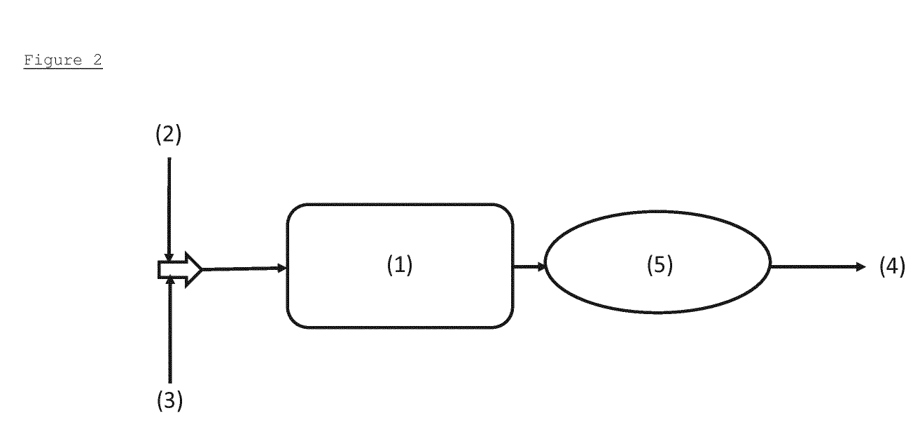

FIG. 2 shows a simplified schematic diagram of the embodiment where two reactor vessels, (1) and (5), are arranged in series. Reactor vessel (1) is a continuous stirred tank reactors and reactor vessel (5) is a plug-flow reactor. The outflow from reactor vessel (1) is supplied to reactor vessel (5) to increase the glycol product levels. Other features of this embodiment, and their respective numbering, are the same as the embodiment described in FIG. 1.

1. A process for the preparation of an unsupported hydrogenation catalyst, wherein a catalyst precursor comprising one or more cations selected from a group consisting of chromium and groups 8, 9, 10 and 11 of the periodic table is contacted with hydrazine in a reactor to convert the catalyst precursor into an unsupported hydrogenation catalyst.

2. The process according to claim 1, wherein the one or more cations is selected from a group consisting of chromium, iron, ruthenium, cobalt, rhodium, iridium, nickel, copper, palladium and platinum.

3. The process according to claim 1, wherein the catalyst precursor comprises an anion selected from a group consisting of carboxylates, acetylacetonate and inorganic anions, which in all cases form a salt or a metal complex that is soluble in a solvent mixture comprising the saccharide-containing feedstock, the solvent and the glycols.

4. The process according to claim 1, wherein the catalyst precursor comprises formate or acetate.

5. The process according to claim 1, wherein the catalyst precursor comprises ruthenium cations.

6. A process for the preparation of glycols from a saccharide-containing feedstock comprising the steps of:
(a) preparing an unsupported hydrogenation catalyst by contacting a catalyst precursor comprising one or more cations selected from a group consisting of chromium and of groups 8, 9, 10 and 11 of the periodic table with hydrazine in a reactor to convert the catalyst precursor into the unsupported hydrogenation catalyst;
(b) preparing in a reactor vessel a reaction mixture comprising the saccharide-containing feedstock, a solvent, a catalyst component with retro-aldol catalytic capabilities and the unsupported hydrogenation catalyst; and
(c) supplying hydrogen gas to the reaction mixture in the reactor vessel.

7. The process according to claim 6, wherein the solvent is water, or a C1, C2, C3, C4, C5 or a C6 alcohol or polyalcohol, or any combination of mixtures thereof.

8. The process according to claim 6, wherein the catalyst component with retro-aldol catalytic capabilities comprises tungsten.

9. The process according to claim 6, wherein the catalyst precursor comprises one or more cations of chromium, iron, ruthenium, cobalt, rhodium, iridium, nickel, copper, palladium and platinum.

10. The process according to claim 6, wherein the catalyst precursor comprises ruthenium cations.

11. The process according to claim 6, wherein the catalyst precursor comprises an anion selected from a group consisting of carboxylates, acetylacetonate and inorganic anions, which in all cases forms a salt or a metal complex that is soluble in a solvent mixture comprising the saccharide-containing feedstock, the solvent and the glycols.

12. The process according to claim 6, wherein the catalyst precursor comprises formate or acetat
